(19) United States
(12) Patent Application Publication
(10) Pub. No.: US 2023/0359791 A1
(43) Pub. Date: Nov. 9, 2023

(54) GENERATING VIRTUAL OPTOELECTRONIC DATA

(71) Applicant: Aesculap AG, Tuttlingen (DE)

(72) Inventors: Michael Utz, Tuttlingen (DE); Allan Maas, Konstanz (DE); Ingrid Dupraz, Tuttlingen (DE); Daniel Alberto Vazquez Urena, Umkirch (DE); Ariana Ortigas Vasquez, Konstanz (DE)

(21) Appl. No.: 18/143,655

(22) Filed: May 5, 2023

(30) Foreign Application Priority Data
May 6, 2022 (DE) 10 2022 111 283.7

(51) Int. Cl. G06F 30/27; G16H 50/30; G16H 50/50

(52) U.S. Cl. CPC G06F 30/27; G16H 50/30; G16H 50/50

(57) ABSTRACT

An electronic device for simulating a joint, in particular a knee joint, and for providing kinematic data sets, includes a simulation unit configured to generate a joint model with at least a first joint bone and a second joint bone. The simulation unit is configured to integrate at least a first and a second virtual measuring unit into the joint model. The at least first virtual measuring unit is at the at least first joint bone and the at least second virtual measuring unit is at the second joint bone. A calculator unit is configured to process data of the at least first and second virtual measuring units.

### CROSS-REFERENCE TO RELATED APPLICATION(S)

This application claims priority under 35 U.S.C. § 119 to German Application No. 10 2022 111 283.7, filed May 6, 2022, the content of which is incorporated by reference herein in its entirety.

### FIELD

The present disclosure relates to a device and a computer-implemented method for simulating a joint or for simulating a movement sequence of a joint in order to generate kinematic data sets and/or to train an AI/ML application.

### BACKGROUND

In order to train AI (artificial intelligence) for clinical applications, a lot of patient data is needed. Collecting or obtaining this data is difficult for various reasons, in particular due to the aspects:

ethics, effort, privacy, patient consent, etc.

As of today, training data for clinical ML (machine learning) applications are collected and recorded with great effort in clinics and with patients.

In the automotive sector, simulations of traffic situations are already used today in order to train neural networks in the field of autonomous driving. This means that the training is not based on real traffic situations, but on their simulation.

### SUMMARY

It is the objective, technical object of the present disclosure to provide a device and/or method with the aid of which as many variants of kinematic data sets of various joints as possible can be generated in a simple and uncomplicated manner.

The aforementioned object is solved by an electronic device for simulating a joint, in particular a knee joint, and for providing kinematic data sets, comprising a simulation unit which is provided and configured to generate a joint model with at least a first joint bone and a second joint bone, wherein the simulation unit is provided and configured to integrate at least a first virtual, preferably inertial, and a second virtual, preferably inertial, measuring unit into the joint model, wherein the at least first virtual measuring unit is arranged at the at least first joint bone and the at least second virtual measuring unit is arranged at the second joint bone, and a calculator unit provided and configured to process data of the at least first and second virtual measuring units. A simulation is the reproduction of things, objects or processes. Thus, the simulation of a joint is the representation of the joint as such as well as the movement of which the joint is capable.

In yet other words, the use of complementary/additional virtual measuring units at the two joint bones forming the joint provides a solution for generating kinematic data sets in the absence of patients, which can be used to train Ms and/or ML applications. This has the technical effect that many data sets with high variance can be generated in a short time with relatively little effort. The virtual measuring units according to the disclosure are preferably configured as virtual inertial measuring units, e.g. as IMUs (inertial measuring units). Alternatively or additionally, they may also be configured as reflective markers that are stuck to the skin or applied via sleeves. In this way, virtual optoelectronic data or position(s) of rigid bodies can be generated. This allows the situation of the soft tissue to be simulated in addition to the bone movement. Another alternative or addition is the creation/generation of data from one or more magnetometer(s) and accelerometer(s) (by measuring acceleration, angular velocity, and/or the magnetic field).

Thus, the present disclosure provides a way to obtain/generate such data in the necessary quantity and with the desired variance by using validated simulation tools/a validated simulation device. Therefore, for example, a kinematic knee model is used to generate theoretical data of the virtual (inertial) measuring units used.

It is advantageous if the device is provided and configured to simulate motion sequences, relative position(s) and an orientation of the at least first joint bone and the second joint bone of different patients.

It is preferred if the device is provided and configured to infer a relative position of the first joint bone to the second joint bone based on data from the at least two virtual measuring units.

It is advantageous if a plurality of parameters and their combinations can be varied and simulated. This offers the possibility to simulate joints of different patients with different problems to and different physical conditions and to obtain data sets.

It is advantageous if the following parameters can be selected or combined with each other as desired:
different load cases, in particular extension and/or flexion with and without load, preferably at different angles of the joint
anatomical variants of the model, in particular varus or valgus or slope or phenotype
patient size and corresponding bone size
patient data, in particular weight and/or BMI and/or soft tissue coverage/situation
position of the virtual measuring units on the joint
orientation of the virtual measuring units

Different load cases include, for example, walking on level ground, climbing stairs and/or, for example, physiotherapy exercises.

In other words, the aforementioned parameters are adjustable and can be combined with each other as desired, whereby a high number of data sets based on different initial situations can be achieved/generated. That is, a large number of the parameters as well as their numerous combinations, can be varied and simulated.

It is preferred if base data/output data of the virtual measuring units is computable during each simulation cycle and the device is provided to output data sets resulting from the base data.

In other words, base data of the virtual (inertial) measuring units are computed at each simulation cycle. By varying the aforementioned parameters, base data of the virtual (inertial) measuring units can be generated for a large number of virtual patients and installations of virtual measuring units/measuring unit setups.

In this context, it is further advantageous if base data for each virtual, preferably inertial measuring unit can be output over time and over flexion and/or extension of the joint. In other words, the base data for each virtual, preferably inertial measuring unit can be output not only over time, as with real inertial measuring units, but the data set can also be output over the flexion and/or extension.

It is advantageous if the data sets resulting from the base data are provided for training neural networks, in particular AI and/or ML applications. In other words, this means that these virtual measuring-unit base data sets are subsequently used for the training of neural networks, with the effect/objective of being able to infer the value progression of the input parameters backwards on the basis of the virtual measuring-unit data.

It is preferred if the device is provided and configured to be extendable by further bony structures, preferably the nearest joint. In other words, the simulation device/model can be extended to include further bony structures. For example, the pelvis may be added to the femur and tibia. This method can also be applied to other joints as described later.

In summary, the simulation device described above can be used to generate many data sets with high variance in a short time with relatively little effort. Here, an essential goal is to be able to infer the relative position of the at least one first joint bone, preferably tibia, to the second joint bone, preferably femur, based on the data obtained by the virtual (inertial) measuring units.

Furthermore, the present disclosure relates to the use of the device according to the aforementioned aspects for hip joint, shoulder joint, elbow joint, knee joint, ankle joint, wrist joint and/or spinal column. In other words, this procedure can in principle be transferred to other, simulatable processes in the human body, in which physiological processes are to be inferred via sensor data.

Furthermore, the present disclosure relates to a computer-implemented method for simulating, training and validating a joint model, in particular a knee joint model, comprising the following steps:
providing a validated, virtual joint model;
integrating virtual (inertial) measuring units;
outputting and processing data sets based on the data of the virtual measuring units;
training an ML application based on the output data sets from the previous step; and
validating the ML application.

In other words, in a first step, possible variant simulations are imported when a validated, virtual joint model is provided. This concerns the setting of parameters, for example the anatomy, the load case, the BMI and/or the soft tissue situation.

In the following step, the virtual (inertial) measuring units are integrated at the first joint bone and the second joint bone. Position, orientation and type are taken into account due to the integration of the virtual measuring units.

In a next step, the acquisition and output of the data obtained/generated from the virtual (inertial) measuring units 5 takes place. This is measuring unit data and/or kinematic data and/or a phenotype derived from the obtained data.

This is followed by a step in which AI/ML applications are trained. The training refers to the kinematics, the phenotype and the relative position as well as orientation of the first joint bone to the second joint bone, e.g. femur to tibia.

In a final step, the AI/ML application has to be validated with respect to the simulation and with respect to at least one patient.

Furthermore, the present disclosure relates to a computer-readable storage medium comprising instructions that, when executed by the aforementioned device, cause the device to perform the method according to the aforementioned aspect.

### BRIEF DESCRIPTION OF THE DRAWINGS

FIG. 1 is an illustration showing a device according to an embodiment of the present disclosure; and

FIG. 2 is an illustration showing a sequence according to an embodiment of the present disclosure.

### DETAILED DESCRIPTION

The following describes configuration examples of the present disclosure based on the accompanying figures.

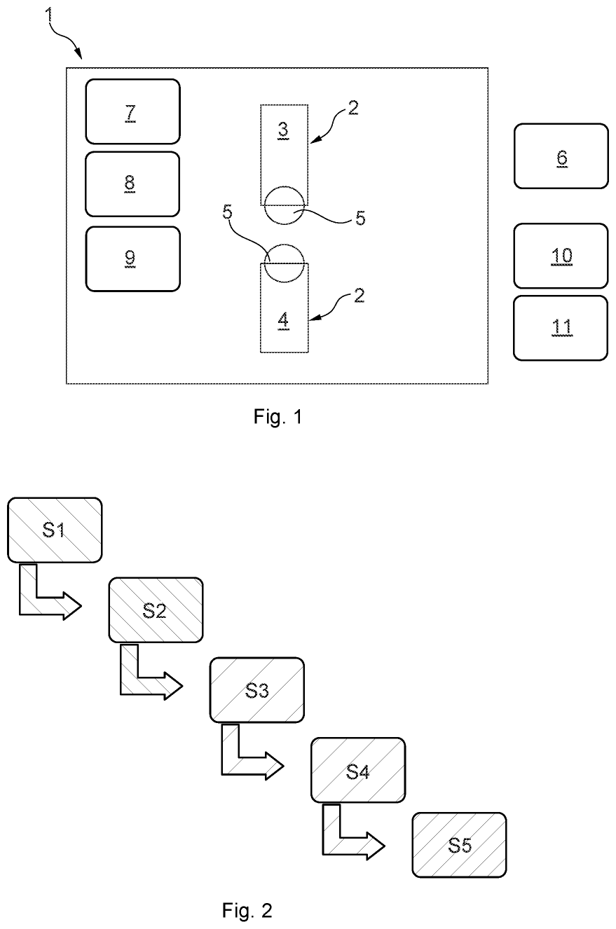

FIG. 1 is an illustration showing a device 1 according to an embodiment of the present disclosure. FIG. 1 shows the electronic device 1 for simulating a joint, in particular a knee joint, and for providing kinematic data sets.

The device 1 has a simulation unit 7, which is provided and configured to generate a joint model 2 with at least a first joint bone 3 and a second joint bone 4. The simulation unit 7 is provided and configured to integrate at least a first and a second virtual, preferably inertial measuring unit 5 into the joint model 2. The at least first virtual, preferably inertial measuring unit 5 is arranged at the at least first joint bone 3 and the at least second virtual, preferably inertial measuring unit 5 is arranged at the second joint bone 4.

FIG. 1 shows a calculator unit 8 and a measuring device 9. The measuring device 9 is provided and configured to integrate and use at least a first and a second measuring unit 5. The calculator unit 8 is provided and configured to process data of at least the first and second virtual, preferably inertial, measuring units 5. The arrangement of the virtual measuring units 5 at the first joint bone 3 and at the second joint bone 4 is merely exemplary. FIG. 1 also shows a training unit 10 and a validation unit 11 which are provided and configured to train and validate neural networks based on the data output of the device 1.

FIG. 2 is an illustration showing a sequence according to an embodiment of the present disclosure. In a first step S1, possible variant simulations are imported when a validated, virtual joint model 2 is provided. This concerns the setting of parameters, for example the anatomy, the load case, the BMI and/or the soft tissue situation.

In a step S2, the virtual, preferably inertial measuring units 5 are integrated at the first joint bone 3 and at the second joint bone 4. Position, orientation and type are taken into account due to the integration of the virtual measuring units 5.

In a next step S3, the acquisition and output of the data obtained/generated from the virtual, preferably inertial measuring units 5 takes place. This is inertial measuring unit data and/or kinematic data and/or a phenotype derived from the obtained data.

This is followed by a step S4 in which the neural network, in particular AI/ML applications 6 is/are trained by means of a training unit 10. The training refers to the kinematics, the phenotype and the relative position as well as orientation of the first joint bone 3 to the second joint bone 4, e.g. femur to tibia.

In a final step S5, the neural network/the AI/ML application 6 has to be validated by means of a validation unit 11 with respect to the simulation and with respect to at least one patient.

1. An electronic device for simulating a joint, and for providing kinematic data sets, comprising:
a simulation unit which is provided and configured to generate a joint model with at least a first joint bone and a second joint bone, wherein the simulation unit is provided and configured to integrate at least a first virtual measuring unit and a second virtual measuring unit into the joint model, wherein the first virtual measuring unit is arranged on the first joint bone and the second virtual measuring unit is arranged on the second joint bone; and
a calculator unit provided and configured to process data of the at least first virtual measuring unit and the second virtual measuring unit.

2. The electronic device according to claim 1, wherein the device is provided and configured to infer a relative position of the first joint bone to the second joint bone based on data from the first virtual measuring unit and the second virtual measuring unit.

3. The electronic device according to claim 1, wherein a plurality of parameters and their combinations are variable or simulatable.

4. The electronic device according to claim 3, wherein the following parameters are selectable or combinable with each other as desired:
different load cases;
anatomical variants of the joint model;
patient size and corresponding bone size;
patient data;
position of the first virtual measuring unit and the second virtual measuring unit on the joint; and
orientation of the first virtual measuring unit and the second virtual measuring unit.

5. The electronic device according to claim 1, wherein base data of the first virtual measuring unit and the second virtual measuring unit is computable during a simulation cycle and the device is provided to output data sets resulting from the base data.

6. The electronic device according to claim 5, wherein the data sets resulting from the base data are provided for training neural networks.

7. The electronic device according to claim 1, wherein base data of the first virtual measuring unit and the second virtual measuring unit is outputted over time and over flexion and/or extension of the joint.

8. The electronic device according to claim 1, wherein the device is provided and configured to extend the simulation of the joint by further bony structures.

9. A method for simulating a joint using the electronic device according to claim 1, comprising the steps of:
generating the joint model with the simulated unit, the joint model being generated with at least the first joint bone and the second joint bone;
integrating at least the first virtual measuring unit and the second virtual measuring unit into the joint model; and
processing data of the at least first virtual measuring unit and the second virtual measuring unit,
wherein the joint is a hip joint, shoulder joint, elbow joint, knee joint, ankle joint, wrist joint and/or spine.

10. A computer-implemented method for simulating a joint model and training and validating neural networks using the electronic device according to claim 1, the method comprising the steps of:
providing a validated, virtual joint model with the electronic device;
integrating the first virtual measuring unit and the second virtual measuring unit into the joint model with a measuring device;
outputting and processing data sets based on data output of the first virtual measuring unit and the second virtual measuring unit with the calculator unit;
training neural networks based on the data sets from the previous step with a training unit; and
validating the neural networks with a validation unit.

11. A computer-readable storage medium comprising instructions for performing the computer-implemented method according to claim 10.
